(19) United States
(12) Patent Application Publication
(10) Pub. No.: US 2026/0108203 A1
(43) Pub. Date: Apr. 23, 2026

(54) METHODS AND SYSTEMS TO MEASURE GASTROINTESTINAL MOTILITY BASED ON A BALLOON BASED SYSTEM

(71) Applicants: THE JOHNS HOPKINS UNIVERSITY, Baltimore, MD (US); The BOARD OF TRUSTEES OF THE LELAND STANFORD JUNIOR UNIVERSITY, Palo Alto, CA (US)

(72) Inventors: Anthony N. Kalloo, Baltimore, MD (US); John O. Clarke, Burlingame, CA (US)

(21) Appl. No.: 19/427,719

(22) Filed: Dec. 19, 2025

Related U.S. Application Data
(63) Continuation of application No. 17/621,087, now abandoned.
(60) Provisional application No. 62/863,547, filed on Jun. 19, 2019.

(51) Int. Cl. A61B 5/00; A61B 1/04

(52) U.S. Cl. CPC A61B 5/4238; A61B 1/041; A61B 5/6843; A61B 5/6853

(57) ABSTRACT

The present invention is directed to a balloon-based system and method for measuring gastrointestinal motility. The balloon can be a stand-alone balloon with pressure sensing capabilities or the balloon can be deployed as a component of a catheter-based system. The balloon can be deployed in an uninflated state and can be inflated with fluid such as air, water, or other biocompatible fluid while disposed within the gastrointestinal tract. The pressure sensor can take the form of a pressure sensor disposed within the balloon or disposed on a surface of the balloon. Additional, sensors can also be applied to monitor other conditions, function, or disease state.

### CROSS REFERENCE TO RELATED APPLICATIONS

This application is a continuation of U.S. patent application Ser. No. 17/621,087, filed on Dec. 20, 2021, which is a 35 U.S.C. § 371 U.S. national entry of International Application PCT/US2020/038609 having an international filing date of Jun. 19, 2020, which claims the benefit of U.S. Provisional Application No. 62/863,547, filed Jun. 19, 2019, the content of each of the aforementioned applications is herein incorporated by reference in their entirety.

### FIELD OF THE INVENTION

The present invention relates generally to medical devices. More particularly, the present invention relates to a method and system to measure gastrointestinal motility based on a balloon based system.

### BACKGROUND OF THE INVENTION

Monitoring gastrointestinal motility can be helpful in the diagnosis and monitoring of several disease states. Generally, gastrointestinal motility is observed with a catheter placed through the nose, mouth, or rectum. While some motility can be observed using this method, it does not allow for the observance and measurement of pressure being applied by the walls of the trachea, intestines, rectum, or other anatomy of the gastrointestinal tract.

Accordingly, there is a need in the art for a method and system to measure gastrointestinal motility based on a balloon based system.

### SUMMARY OF THE INVENTION

The foregoing needs are met, to a great extent, by the present invention which provides a device for sensing pressure in the gastrointestinal tract including a balloon. The device also includes a pressure sensor. The pressure sensor is configured to record and wirelessly transmit data regarding pressures measured in the gastrointestinal tract.

In accordance with an aspect of the present invention, the pressure is disposed within the balloon or on a surface of the balloon. The balloon is configured to be inflated by a catheter, a syringe, or a chemical reaction that causes expansion of the balloon. The balloon can also be configured to be swallowed. The device can include additional sensors configured to report data from within the gastrointestinal tract.

In accordance with another aspect of the present invention, a system for sensing pressure in the gastrointestinal tract includes a catheter. A balloon is configured to be disposed within the catheter when the balloon is in an uninflated state. The system includes a pressure sensor. The pressure sensor is configured to record and wirelessly transmit data regarding pressures measured in the gastrointestinal tract. The balloon is configured to be inflated by the catheter upon release into the gastrointestinal tract. The balloon is configured to be inflated by a second catheter. The balloon is configured to be inflated by a syringe. The balloon is configured to be inflated by a chemical reaction that causes expansion of the balloon. The balloon is configured to be deployed in an uninflated state and can be inflated with fluid such as air, water, or other biocompatible fluid while disposed within the gastrointestinal tract.

In accordance with yet another aspect of the present invention, the pressure sensor can be disposed within the balloon, or the pressure sensor being disposed on a surface of the balloon. Alternately, there can be pressure sensors positioned on both the surface and disposed within the balloon. The pressure sensor includes a first pressure sensor disposed within the balloon and a second pressure sensor disposed on a surface of the balloon. The system can also include additional sensors configured to report data from within the gastrointestinal tract. The balloon is formed from a thermoplastic elastomer.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a partially sectional view of a balloon with internal pressure sensor, according to an embodiment of the present invention.

FIG. 2 illustrates a side view of a balloon with external pressure sensor, according to an embodiment of the present invention.

FIGS. 3 and 4 illustrate views of a balloon, according to an embodiment of the present invention, disposed within the gastrointestinal tract.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a balloon-based system and method for measuring gastrointestinal motility. The balloon can be a stand-alone balloon with pressure sensing capabilities or the balloon can be deployed as a component of a catheter-based system. The balloon can be deployed in an uninflated state and can be inflated with fluid such as air, water, or other biocompatible fluid while disposed within the gastrointestinal tract. The pressure sensor can take the form of a pressure sensor disposed within the balloon or disposed on a surface of the balloon. Additional, sensors can also be applied to monitor other conditions, function, or disease state.

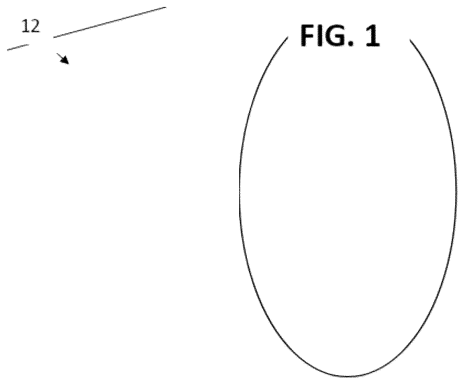

FIG. 1 illustrates a partially sectional view of a balloon with internal pressure sensor, according to an embodiment of the present invention. The balloon 10 can be inflated within the gastrointestinal system. In some embodiments, the balloon can be deployed with a catheter that can also act to inflate the balloon with a fluid, after it has been deployed. The balloon 10 can also be deployed independently and inflated, after it has been deployed. In such an embodiment, the balloon might contain materials that react to inflate the balloon, after it has been deployed. The balloon can be filled with a variable or predetermined amount of fluid. In embodiments where the balloon is being deployed to measure pressure in the colon or rectum, it is also possible for the balloon to be inflated by a syringe filled with fluid. The pressure sensor 12 can be disposed within the balloon 10. When pressure is applied to the balloon by the gastrointestinal system, pressure is thereby also applied to the pressure sensor 12 within the balloon 10. The pressure sensor 12 can be configured to communicate and transmit data to a computing device or telemetric device, wirelessly. This wireless communication can take place via wifi, Bluetooth™, RFID, or other wireless communication modality known to or conceivable by one of skill in the art. A non-transitory computer readable medium can be programmed to process the data transmitted by the pressure sensor 12. The balloon can be formed from a thermoplastic elastomer or other biocompatible and inflatable material. The device can also include additional sensors known to or conceivable to one of skill in the art for measuring other physiologic conditions.

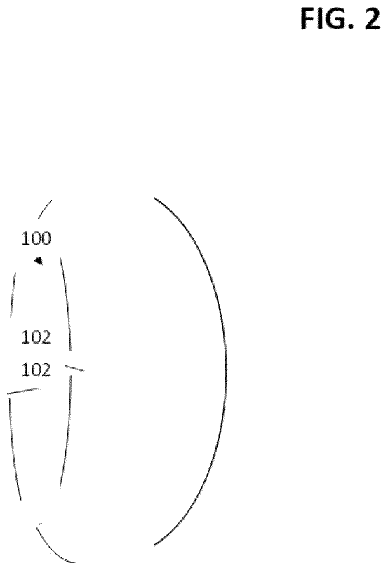

FIG. 2 illustrates a side view of a balloon with external pressure sensor, according to an embodiment of the present invention. The balloon 100 can be inflated within the gastrointestinal system. In some embodiments, the balloon can be deployed with a catheter that can also act to inflate the balloon with a fluid, after it has been deployed. The balloon 100 can also be deployed independently and inflated, after it has been deployed. In such an embodiment, the balloon might contain materials that react to inflate the balloon, after it has been deployed. In embodiments where the balloon is being deployed to measure pressure in the colon or rectum, it is also possible for the balloon to be inflated by a syringe filled with fluid. The pressure sensor 102 can be disposed on an outside surface of the balloon 100. When pressure is applied to the balloon by the gastrointestinal system, pressure is thereby also applied to the pressure sensor 102. The pressure sensor 102 can take the form of pressure sensing strips, as illustrated in FIG. 2. Alternately, the balloon 100 can be covered in individual pressure sensors 102. The pressure sensor 12 can be configured to communicate and transmit data to a computing device wirelessly. This wireless communication can take place via wifi, Bluetooth™, RFID, or other wireless communication modality known to or conceivable by one of skill in the art. A non-transitory computer readable medium can be programmed to process the data transmitted by the pressure sensor 102. The balloon can be formed from a thermoplastic elastomer or other biocompatible and inflatable material.

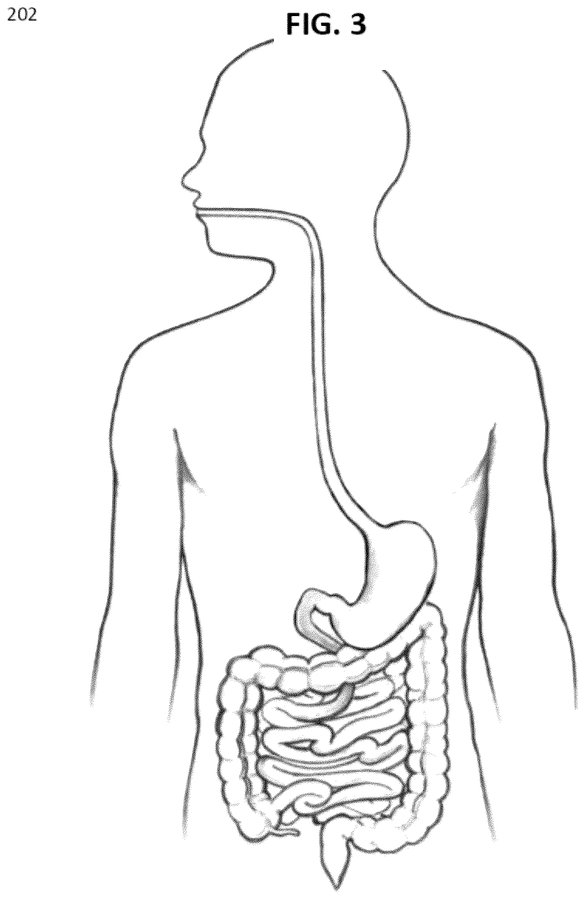

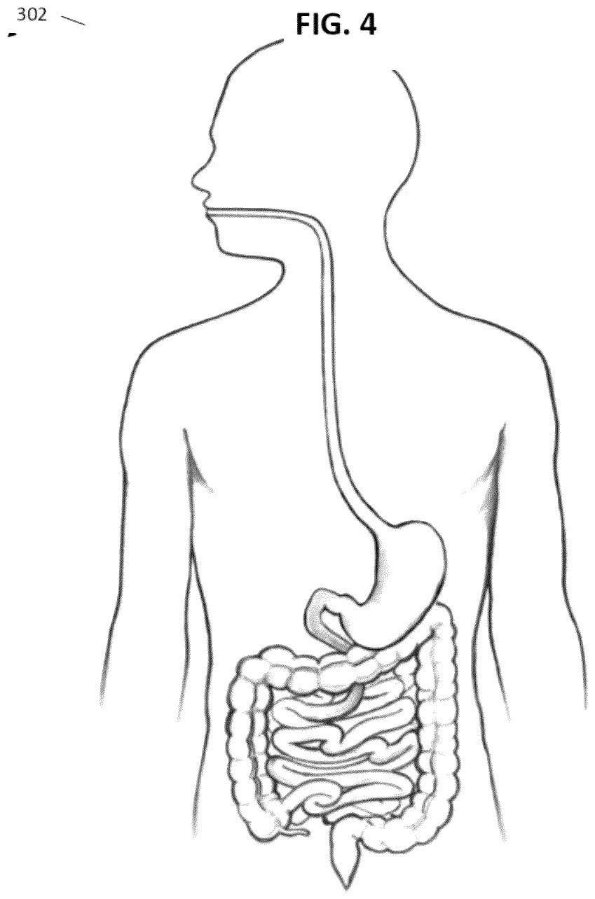

FIGS. 3 and 4 illustrate views of a balloon, according to an embodiment of the present invention, disposed within the gastrointestinal tract. To evaluate the upper gastrointestinal tract 200 and swallowing mechanisms, the balloon 202 can be swallowed, as illustrated in FIG. 4. To evaluate anorectal function, the balloon 302 is inserted into the rectum of the gastrointestinal tract 300 and inflated to various sizes, for instance using a catheter. Basal rectal pressure is recorded. The patient is then asked to expel the balloon, simulating defecation. The balloon 302 moves through the rectum and measures pressure in real time during the process of being expelled. The pressures generated during this process are recorded and used to diagnose conditions that affect anorectal function. Data is transmitted by the pressure sensor in either embodiment to a computing device or telemetric device. A non-transitory computer readable medium is programmed to process the data transmitted by the pressure sensor.

The present invention carried out using a computer, non-transitory computer readable medium, or alternately a computing device or non-transitory computer readable medium incorporated into a console for deployment and/or visualization of the balloon. Indeed, any suitable method of calculation known to or conceivable by one of skill in the art could be used. It should also be noted that to the extent specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. It is not a standard business or personal computer that can be purchased at a local store. Additionally this computer carries out communications with the pressure sensing devices through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A device for sensing pressure in the gastrointestinal tract, comprising:
a balloon; and,
a pressure sensor;
wherein the pressure sensor is configured to record and wirelessly transmit data regarding pressures measured in the gastrointestinal tract.

2. The device of claim 1 further comprising the pressure sensor being disposed within the balloon.

3. The device of claim 1 further comprising the pressure sensor being disposed on a surface of the balloon.

4. The device of claim 1 wherein the pressure sensor comprises a first pressure sensor disposed within the balloon and a second pressure sensor disposed on a surface of the balloon.

5. The device of claim 1 wherein the balloon is configured to be inflated by a catheter.

6. The device of claim 1 wherein the balloon is configured to be inflated by a syringe.

7. The device of claim 1 wherein the balloon is configured to be inflated by a chemical reaction that causes expansion of the balloon.

8. The device of claim 1 wherein the balloon is configured to be swallowed.

9. The device of claim 1 further comprising additional sensors configured to report data from within the gastrointestinal tract.

10. A system for sensing pressure in the gastrointestinal tract, comprising:
a catheter;
a balloon configured to be disposed within the catheter when the balloon is in an uninflated state;
a pressure sensor;
wherein the pressure sensor is configured to record and wirelessly transmit data regarding pressures measured in the gastrointestinal tract.

11. The system of claim 10 wherein the balloon is configured to be inflated by the catheter upon release into the gastrointestinal tract.

12. The system of claim 10 wherein the balloon is configured to be inflated by a second catheter.

13. The system of claim 10 wherein the balloon is configured to be inflated by a syringe.

14. The system of claim 10 wherein the balloon is configured to be inflated by a chemical reaction that causes expansion of the balloon.

15. The system of claim 10 wherein the balloon is configured to be deployed in an uninflated state and can be inflated with fluid such as air, water, or other biocompatible fluid while disposed within the gastrointestinal tract.

16. The system of claim 10 further comprising the pressure sensor being disposed within the balloon.

17. The system of claim 10 further comprising the pressure sensor being disposed on a surface of the balloon.

18. The system of claim 10 wherein the pressure sensor comprises a first pressure sensor disposed within the balloon and a second pressure sensor disposed on a surface of the balloon.

19. The system of claim 10 further comprising additional sensors configured to report data from within the gastrointestinal tract.

20. The system of claim 10 wherein the balloon is formed from a thermoplastic elastomer.
